(19) United States
(12) Patent Application Publication
(10) Pub. No.: US 2025/0134409 A1
(43) Pub. Date: May 1, 2025

(54) COUGH MONITORING

(71) Applicant: STMicroelectronics International N.V., Geneva (CH)

(72) Inventors: Federico RIZZARDINI, Settimo Milanese (IT); Alessandro MAGNANI, Milan (IT)

(73) Assignee: STMicroelectronics International N.V., Geneva (CH)

(21) Appl. No.: 18/496,728

(22) Filed: Oct. 27, 2023

(51) Int. Cl. A61B 5/08; A61B 5/00; A61B 5/11

(52) U.S. Cl. CPC A61B 5/0823; A61B 5/11; A61B 5/7221; A61B 5/7225; A61B 5/7246; A61B 5/7257; A61B 5/7267; A61B 5/7282; A61B 5/6803; A61B 2560/02; A61B 2560/0462; A61B 2562/0204; A61B 2562/0219

(57) ABSTRACT

The present disclosure is directed to cough detection for electronic devices, such as wireless headphones. The cough detection utilizes inertial sensors to perform both head movement detection and vocal activity detection. The dual identification of head movement and vocal activity allows improved detection accuracy, and minimal false detections caused by environmental noise.

### BACKGROUND

### Technical Field

The present disclosure is directed to cough monitoring for electronic devices.

### Description of the Related Art

Many electronic devices include cough detection to provide real-time health data of users. For example, cough detection may be used to track coughing patterns to aid health care professionals diagnose several diseases, such as pulmonary infections.

Cough detection is typically implemented in ad hoc devices positioned near the part of the body generating the signal, such as a wearable chest band positioned at a user's chest. These devices typically rely on audio signals detected by microphones.

Due to size and inconvenient nature of such devices, these devices are usually worn by the user on an as needed basis. Further, microphone signals are generally susceptible to noise and external disturbances. As a result, current cough detection algorithms are complex with high computational processing costs to ensure accurate detection results.

### BRIEF SUMMARY

The present disclosure is directed to cough detection for electronic devices, such as earphones, headphones, eyeglasses, and other types of wearable devices. The cough detection includes both head movement detection and vocal activity detection. Head movement detection and vocal activity detection utilize acceleration measurements generated by, for example, a bone conduction accelerometer. In contrast to microphones, bone conduction accelerometers are capable of sensing the sound/vibration propagated through human bones, and, thus, are not susceptible to noise and subject to external acoustic disturbances.

The head movement detection detects head movement that is consistent with a cough, such as sudden head movement forward and/or backward. Head movement is detected based on acceleration measurements in the time domain.

The vocal activity detection detects vocal activity consistent with a cough, such as a loud and short vocal sound. Vocal activity is detected based on acceleration measurements in the frequency domain.

A cough is detected when both the head movement and voice activity consistent with a cough is detected, along with timing constraints being satisfied. The dual identification of head movement and vocal activity allows precise cough detection with minimal false positives and negatives. In addition, in contrast to a microphone, the accelerometer is largely immune to noise and external acoustic disturbances, and has much lower power consumption.

### BRIEF DESCRIPTION OF THE SEVERAL VIEWS OF THE DRAWINGS

In the drawings, identical reference numbers identify similar features or elements. The size and relative positions of features in the drawings are not necessarily drawn to scale.

FIG. 1 is a device according to an embodiment disclosed herein.

FIG. 2 is a flow diagram of a method for performing cough detection according to an embodiment disclosed herein.

FIG. 3 is a flow diagram of pre-conditioning in the method of FIG. 2 according to an embodiment disclosed herein.

FIG. 4 is a flow diagram of a method for performing cough detection according to another embodiment disclosed herein.

### DETAILED DESCRIPTION

In the following description, certain specific details are set forth in order to provide a thorough understanding of various aspects of the disclosed subject matter. However, the disclosed subject matter may be practiced without these specific details. In some instances, well-known machine learning techniques and structures, functions, and methods of manufacturing electronic devices, electronic components, and sensors have not been described in detail to avoid obscuring the descriptions of other aspects of the present disclosure.

Unless the context requires otherwise, throughout the specification and claims that follow, the word “comprise” and variations thereof, such as “comprises” and “comprising.” are to be construed in an open, inclusive sense, that is, as “including, but not limited to.”

Reference throughout the specification to “one embodiment” or “an embodiment” means that a particular feature, structure, or characteristic described in connection with the embodiment is included in at least one embodiment. Thus, the appearance of the phrases “in one embodiment” or “in an embodiment” in various places throughout the specification are not necessarily all referring to the same aspect. Furthermore, the particular features, structures, or characteristics may be combined in any suitable manner in one or more aspects of the present disclosure.

As discussed above, cough detection is typically implemented in devices positioned near the part of the body generating the signal, and rely on audio signals detected by microphones. Due to size and inconvenient nature of such devices, these devices are usually worn by the user on an as needed basis. Further, microphone signals are susceptible to noise and subject to external acoustic disturbances.

The present disclosure is directed to cough detection based on inertial sensors. The cough detection is implemented in an electronic device worn on a user's ear, such as wireless headphones. Rather than a microphone, the cough detection utilizes an accelerometer, such as a bone conduction accelerometer, that is capable of sensing the sound/vibration propagated through human bones. The accelerometer is able to detect head movements and vocal activity that are consistent with a cough event. The accelerometer is capable of detecting cough profiles similar to microphones due to wide-bandwidth of the accelerometer. The dual identification of head movement and vocal activity allows precise cough detection with minimal false positives and negatives. In addition, in contrast to a microphone, the accelerometer is largely immune to noise and external acoustic disturbances, and has much lower power consumption.

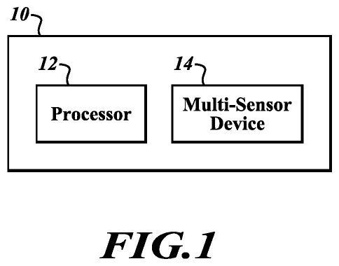

FIG. 1 is a device 10 according to an embodiment disclosed herein.

The device 10 is an electronic device that is configured to perform cough detection. The device 10 is a device that is worn by a user, such as earphones, headphones, and eyeglasses. The device 10 includes a processor 12 and a multi-sensor device 14. The device 10 may include various other components, such as speakers, microphones, proximity sensors, batteries, etc.

The processor 12 is a general-purpose processor that performs various functions for the device 10. For example, the processor 12 executes various applications, controls and coordinates hardware components of the device 10, and communicates with any peripheral devices communicatively coupled to the device 10. The processor 12 may include one or more processors.

The multi-sensor device 14 is communicatively coupled to the processor 12. The multi-sensor device 14 includes one or more types of motion sensors including, but not limited to, an accelerometer and a gyroscope that generate motion measurements. The accelerometer and the gyroscope measure acceleration and angular velocity or rate, respectively, along one or more axes. In one embodiment, the accelerometer is a bone conduction accelerometer that is capable of sensing the sound/vibration propagated through human bones.

The multi-sensor device 14 also includes its own onboard memory, and a processor or processing circuitry coupled to the onboard memory. The processor is configured to receive and process data generated by the sensors; and execute programs stored in the onboard memory. The processor may include one or more processors.

In contrast to a general-purpose processor like the processor 12, the multi-sensor device 14 is a power-efficient, low-powered device, such as a smart sensor, that consumes between, for example, 100 and 300 microamps for computational requirements during processing. As such, battery life of the device 10 is improved in case the power source is a rechargeable battery.

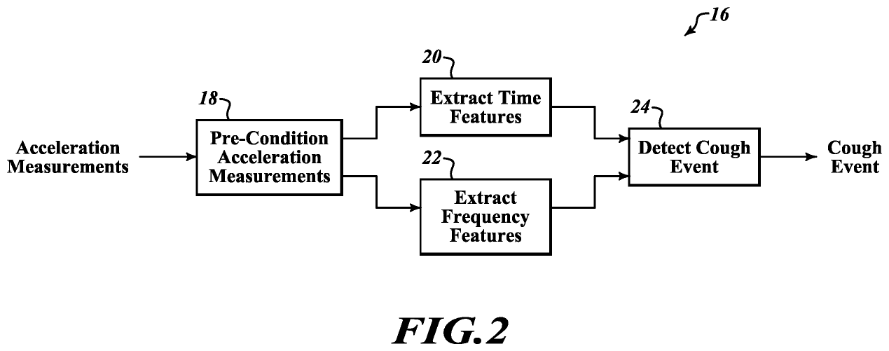

FIG. 2 is a flow diagram of a method 16 for performing cough detection according to an embodiment disclosed herein.

The method 16 is executed by the device 10. More specifically, the method 16 is implemented as a program or a set of instructions that can be downloaded and stored in the onboard memory of the multi-sensor device 14, and is executed by the processor included in the multi-sensor device 14. It is also possible for the program for the method 16 to be stored in memory of the device 10, and executed by processor 12 of the device 10.

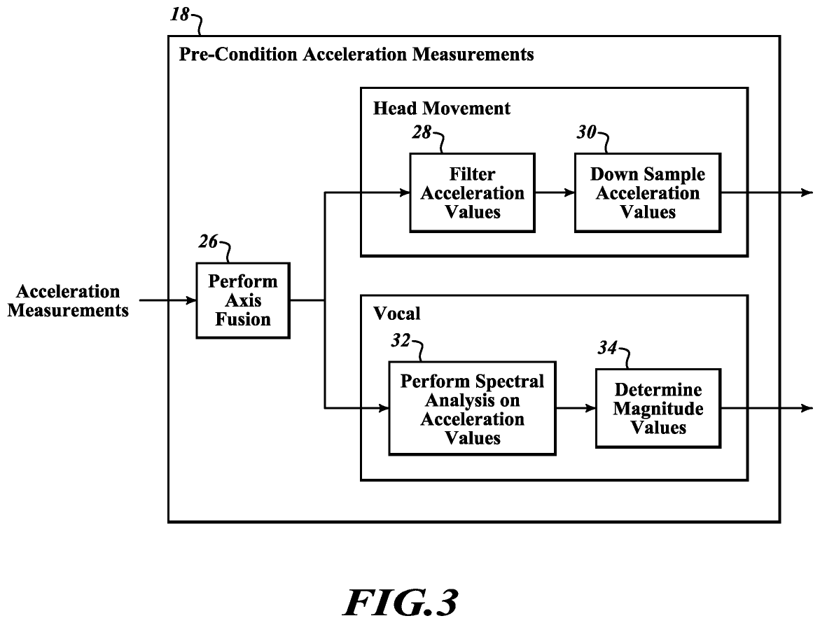

In block 18, acceleration measurements are generated by the accelerometer included in the multi-sensor device 14, and pre-conditioned for head movement detection and vocal detection. FIG. 3 is a flow diagram of the pre-conditioning in block 18 according to an embodiment disclosed herein.

In block 26, in case acceleration is measured along multiple axes (e.g., three axes), the acceleration measurements are fused to obtain acceleration values av indicative of acceleration along the multiple axes, without corruptions or erasing correlation between axes. For example, the acceleration values av may be calculated using any one of following equations:

$$av = \sqrt{{x}^{2} + {y}^{2} + {z}^{2}} \\ av = x + y + z \\ av = Polar Rotation of acceleration measurements$$

where x is acceleration along an x-axis, y is acceleration along a y-axis transverse to the x-axis, and z is acceleration along a z-axis transverse to the x-axis and the y-axis. Other types of fusing calculations are also possible, such as principal component analysis (PCA).

In another embodiment, in case acceleration is measured along multiple axes (e.g., three axes), acceleration measurements along one or more axes is selected as the acceleration values av. For example, acceleration values along one of the x-axis, y-axis, or z-axis is selected.

The acceleration values av are then separately processed for head movement detection and vocal detection. Blocks 28 and 30 are for head movement detection, and blocks 32 and 34 are for vocal detection.

In block 28, the acceleration values av are filtered with a band pass filter to obtain low frequencies indicative of head movements. In one embodiment, the band pass filter cuts off frequencies below 1 hertz and greater than 100 hertz.

In block 30, the filtered acceleration values av are down sampled to obtain sufficient samples to detect head movements and for ease of processing. Block 30 is optional and may be removed in some cases. The filtered and down sampled acceleration values av are then output to block 20, which will be discussed in further detail below.

In block 32, spectral analysis is performed on the acceleration values av in order to characterize the acceleration values in the frequency domain, and detect middle-high frequencies (e.g., greater than 85 hertz) caused by vocal activity. In one embodiment, a Fast Fourier Transform (FFT), a Sliding Discrete Fourier Transform (SDFT), or a similar technique, is performed on adjacent time-windows to convert the acceleration values av to the frequency domain. Vocal activity appears as tiny, high-energy rows on the spectrogram.

In block 34, magnitude values of the acceleration values av in the frequency domain are determined. The magnitude values are then output to block 22, which will be discussed in further detail below.

Returning to FIG. 2, in block 20, time features or characteristics of the filtered and down sampled acceleration values av from block 30 are determined. The time features characterize the acceleration signal information of the acceleration values av in the time domain.

The features may include, for example, one or more of the following calculations: a root mean square calculation (e.g., the root mean square of acceleration values av in a period of time), an auto correlation calculation (e.g., the auto correlation of acceleration values av in a period of time), an envelope calculation (e.g., the envelope of acceleration values av in a period of time), and a zero crossing rate calculation (e.g., the zero crossing rate of acceleration values av in a period of time). Other types of calculations are also possible. In one embodiment, the root mean square calculation is performed in block 20. The time features are then output to block 24, which will be discussed in further detail below.

In block 22, frequency features of the magnitude values from block 34 are determined. Block 22 may be performed concurrently with block 20. The frequency features characterize the acceleration signal information of the magnitude values in the frequency domain.

The features may include, for example, one or more of the following calculations: an energy calculation (e.g., the sum of bins of acceleration values av in a frequency range), a spectral centroid calculation (e.g., the spectral centroid of bins of acceleration values av in a frequency range), a spectral spread calculation (e.g., the spectral spread of bins of acceleration values av in a frequency range), and a maximum energy bin calculation (e.g., the maximum energy bin or frequency peak of acceleration values av in a frequency range). Other types of calculations are also possible. In one embodiment, the maximum energy bin calculation is performed in block 22. The frequency features are then output to block 24.

In block 24, a cough event is detected based on the time features from block 20 and the frequency features from block 22. A cough event is detected in response to (1) one or more time features satisfying determined time feature conditions, (2) one or more frequency features satisfying determined frequency feature conditions, and (3) one or more event timing constraints satisfying determined event timing conditions. Conversely, no cough event is detected in response to (1) the one or more time features not satisfying determined time feature conditions, (2) the one or more frequency features not satisfying determined frequency feature conditions, or (3) the one or more event timing constraints not satisfying determined event timing conditions.

The time features satisfying determined time feature conditions is indicative of head movement consistent with a cough, such as sudden head movement forward and/or backward. The frequency features satisfying determined frequency feature conditions is indicative of vocal activity consistent with a cough, such as a loud and short vocal sound. The event timing constraints satisfying determined event timing conditions is indicative of timings of head movement and vocal activity that are consistent with a cough.

In further detail, a preliminary cough event is detected in case (1) one or more time features satisfies determined time feature conditions, and (2) one or more frequency features satisfies determined frequency feature conditions. For example, in case the time features in block 20 include a root mean square calculation and the frequency features in block 22 include a maximum energy bin calculation, a preliminary cough event is detected in case (1) the root mean square calculation is greater than a determined threshold and (2) the maximum energy bin calculation is greater than a first determined threshold and less than a second determined threshold greater than the first threshold. As discussed above, time features may include one or more of a root mean square calculation, an auto correlation calculation, an envelope calculation, and a zero crossing rate calculation; and frequency features may include one or more of an energy calculation, a spectral centroid calculation, a spectral spread, and a maximum energy bin calculation.

The preliminary cough event is validated as a cough event in case one or more event timing constraints satisfy determined event timing conditions. Timing constraints include, for example, a time duration of the preliminary cough event and a time distance from a previous preliminary cough event. For example, the preliminary cough event is validated as a cough event in case (1) the time duration of the preliminary cough event is greater than a first determined threshold (e.g., 30 milliseconds) and less than a second determined threshold greater than the first threshold (e.g., 200 milliseconds), and (2) the time distance from a previous cough event is greater than a determined threshold (e.g., 200 milliseconds). Conversely, the preliminary cough event is invalidated as a false cough event in case (1) the time duration of the preliminary cough event is not between the first determined threshold and the second determined threshold, or (2) the time distance from a previous preliminary cough event is not greater than the determined threshold.

A cough event is output for further processing in response to the preliminary cough event being validated. For example, the cough may be logged along with a time stamp of the cough event, and further processed with previous cough events to determine a coughing pattern. The method 16 is then repeated in order to continue to perform cough detection.

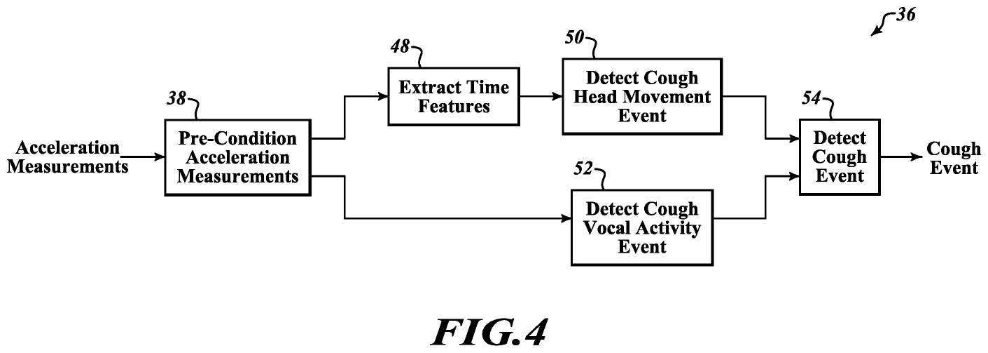

FIG. 4 is a flow diagram of a method 36 for performing cough detection according to another embodiment disclosed herein. In contrast to the method 16 of FIG. 2, the method 36 utilizes machine learning techniques. The use of machine learning techniques improves detection accuracy, and minimizes false detections caused by other types of head movements and vocal sounds.

The method 36 is executed by the device 10. More specifically, the method 36 is implemented as a program or a set of instructions that can be downloaded and stored in the onboard memory of the multi-sensor device 14, and is executed by the processor included in the multi-sensor device 14. It is also possible for the program for the method 36 to be stored in memory of the device 10, and executed by processor 12 of the device 10.

In block 38, acceleration measurements are generated by the accelerometer included in the multi-sensor device 14, and pre-conditioned for head movement detection and vocal detection. The acceleration measurements are pre-conditioned as discussed with respect to FIG. 3.

As discussed above, in block 38, in case acceleration is measured along multiple axes (e.g., three axes), acceleration measurements are fused to obtain acceleration values av indicative of acceleration along the multiple axes. Alternatively, acceleration measurements along one or more axes is selected.

The acceleration values av are then separately processed for head movement detection and vocal detection. Blocks 28 and 30 are for head movement detection, and blocks 32 and 34 are for vocal detection.

In block 28, the acceleration values av are filtered with a band pass filter to obtain low frequencies indicative of head movements.

In block 30, the filtered acceleration values av are then down sampled to obtain sufficient samples to detect head movements and for ease of processing. The filtered and down sampled acceleration values av are then output to block 48, which will be discussed in further detail below.

In block 32, spectral analysis is performed on the acceleration values av in order to characterize the acceleration values in the frequency domain, and detect middle-high frequencies (e.g., greater than 85 hertz) caused by vocal activity.

In block 34, magnitude values of the acceleration values av in the frequency domain are determined. The magnitude values are then output to block 52, which will be discussed in further detail below.

Returning to FIG. 4, in block 48, time features of the filtered and down sampled acceleration values av from block 30 are determined. As discussed above with respect to block 20 of FIG. 2, the time features characterize the acceleration signal information of the acceleration values av in the time domain.

The features may include, for example, one or more of the following calculations: a root mean square calculation (e.g., the root mean square of acceleration values av in a period of time), an auto correlation calculation (e.g., the auto correlation of acceleration values av in a period of time), an envelope calculation (e.g., the envelope of acceleration values av in a period of time), and a zero crossing rate calculation (e.g., the zero crossing rate of acceleration values av in a period of time). Other types of calculations are also possible. In one embodiment, the envelope calculation is performed in block 48. The time features are then output to block 50.

In block 50, machine learning techniques are used to classify the time features in block 48 as head movement consistent with a cough or head movement inconsistent with a cough. A cough head movement event is detected in response to classifying the time features as head movement consistent with a cough. For example, in case the time features in block 48 include an envelope calculation, a cough head movement event is detected in response to the envelope being classified as head movement consistent with a cough. The cough head movement event is output to block 54, which will be discussed in further detail below.

Machine learning techniques include at least one of a decision tree, a neural network, and a support vector machine. Other machine learning techniques are also possible.

Learning/inference machines may fall under the technological titles of machine learning, artificial intelligence, artificial neural networks (ANN), probabilistic inference engines (e.g., Markov models), and the like. One approach is to collect and label a set of training acceleration data or features computed starting from the acceleration data (e.g., envelope calculation) for cough head movement with the purpose of training a supervised machine learning model. Classification problems, such as cough detection, and other signal processing applications, particularly benefit from the use of learning/inference machines, such as convolutional neural networks (CNN), fuzzy-logic machines, etc. For example, a CNN is a computer-based tool that processes large quantities of data, such as sensor data, and adaptively “learns” by conflating proximally related features within the data, making broad predictions about the data, and refining the predictions based on reliable conclusions and new conflations. The CNN is arranged in a plurality of “layers,” and different types of predictions are made at each layer.

In one embodiment, the time features in block 48 includes an envelope calculation, and the envelope is classified as head movement consistent with a cough or head movement inconsistent with a cough in block 50 with a one-dimensional CNN. Other techniques, such as utilizing recurrent neurons (e.g., Long Short-Term Memory (LSTM), Gated Recurrent Unit (GRU), etc.) may also be used.

In block 52, machine learning techniques are used to classify the magnitude values in block 34 as vocal activity consistent with a cough or vocal activity inconsistent with a cough. Block 52 may be performed concurrently with blocks 48 and 50. A cough vocal activity event is detected in response to classifying the magnitude values as vocal activity consistent with a cough. The cough vocal activity event is output to block 54, which will be discussed in further detail below.

As discussed above, machine learning techniques include at least one of a decision tree, a neural network, and a support vector machine. Other machine learning techniques are also possible.

In one embodiment, the magnitude values are classified as vocal activity consistent with a cough or vocal activity inconsistent with a cough in block 52 with a two-dimensional CNN in order to better identify tiny, high-energy rows on the spectrogram. Other techniques, such as utilizing recurrent neurons (e.g., Long Short-Term Memory (LSTM), Gated Recurrent Unit (GRU), etc.) may also be used.

In block 54, a cough event is detected in response to (1) a cough head movement event being detected in block 50, (2) a vocal activity event being detected in block 52, and (3) one or more event timing constraints satisfying determined event timing conditions. Conversely, no cough event is detected in response to (1) a cough head movement event not being detected in block 50, (2) a vocal activity event not being detected in block 52, or (3) the one or more event timing constraints not satisfying determined event timing conditions.

In further detail, a preliminary cough event is detected in case (1) a cough head movement event is detected in block 50, and (2) a vocal activity event is detected in block 52. The preliminary cough event is validated as a cough event in case one or more event timing constraints satisfy determined event timing conditions. As discussed above, timing constraints include, for example, a time duration of the preliminary cough event and a time distance from a previous preliminary cough event.

A cough event is output for further processing in response to the preliminary cough event being validated. For example, the cough may be logged along with a time stamp of the cough event, and further processed with previous cough events to determine a coughing pattern. The method 36 is then repeated in order to continue to perform cough detection.

The various embodiments disclosed herein provide cough detection for electronic devices, such as wireless headphones. The cough detection includes both head movement detection and vocal activity detection. The dual identification of head movement and vocal activity allows precise cough detection with minimal false positives and negatives. In addition, in contrast to a microphone, the accelerometer is largely immune to noise.

A device may be summarized as including: a motion sensor configured to generate acceleration measurements along a plurality of axes; and a processor coupled to the motion sensor, the processor configured to: detect a head movement event based on the acceleration measurements, the head movement event indicating cough head movement by a user; detect a vocal activity event based on the acceleration measurements, the vocal activity event indicating cough vocal activity by the user; detect a cough event in response to detection of the head movement event and the vocal activity event; and output the cough event.

The device may be a multi-sensor device that includes the motion sensor and the processor.

The motion sensor may include a bone conduction accelerometer configured to generate the acceleration measurements.

The processor may be configured to: determine acceleration values based on the acceleration measurements, the acceleration values being indicative of the acceleration measurements along the plurality of axes, the head movement event being detected based on the acceleration values, the vocal activity event being detected based on the acceleration values.

The processor may be configured to: filter the acceleration values with a band pass filter; and down sample the filtered acceleration values.

The processor may be configured to: determine time domain features of the down sampled filtered acceleration values; and detect the head movement event in case the time domain features satisfy one or more determined conditions.

The time domain features may include at least one of a root mean square calculation, an auto correlation calculation, an envelope calculation, and a zero crossing rate calculation.

The time domain features may include a root mean square calculation.

The processor may be configured to: convert the acceleration values to a frequency domain; and determine magnitude values of the acceleration values in the frequency domain.

The processor may be configured to: determine frequency domain features of the magnitude values of the acceleration values in the frequency domain; and detect the vocal activity event in case the frequency domain features satisfy one or more determined conditions.

The frequency domain features may include at least one of an energy calculation, a spectral centroid calculation, a spectral spread calculation, and a maximum energy bin calculation.

The frequency domain features may include a maximum energy bin calculation.

The processor may be configured to: validate the cough event in case the cough event is detected for a determined time duration and detected a determined amount of time after a previous cough event.

The processor may be configured to: determine acceleration values based on the acceleration measurements; filter the acceleration values along the axis with a band pass filter; down sample the filtered acceleration values; determine time domain features of the down sampled filtered acceleration values; and detect the head movement event based on the time domain features.

The time domain features may include an envelope calculation, and the head movement event may be detected with a neural network.

The processor may be configured to: determine acceleration values based on the acceleration measurements; convert the acceleration values along the axis to a frequency domain; determine magnitude values of the acceleration values in the frequency domain; and detect the vocal activity event based on the magnitude values.

The vocal activity may be detected with a neural network.

A method may be summarized as including: generating, by a motion sensor, acceleration measurements along a plurality of axes; detecting, by a processor coupled to the motion sensor, a head movement event based on the acceleration measurements, the head movement event indicating cough head movement by a user; detecting, by the processor, a vocal activity event based on the acceleration measurements, the vocal activity event indicating cough vocal activity by the user; detecting, by the processor, a cough event in response to detecting the head movement event and the vocal activity event; and outputting, by the processor, the cough event.

The method may further include: validating, by the processor, the cough event in case the cough event is detected for a determined time duration and detected a determined amount of time after a previous cough event.

A multi-sensor device may be summarized as including: an accelerometer configured to generate acceleration measurements; and a processor coupled to the accelerometer, the processor configured to: determine time domain features of the acceleration measurements; detect a head movement event based on the time domain features, the head movement event indicating cough head movement by a user; convert the acceleration measurements to a frequency domain; detect a vocal activity event based on the acceleration measurements in the frequency domain, the vocal activity event indicating cough vocal activity by the user; detect a cough event in response to detection of the head movement event and the vocal activity event; and output the cough event.

The processor may be configured to: validate the cough event in case the cough event is detected for a determined time duration and detected a determined amount of time after a previous cough event.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A device, comprising:
a motion sensor configured to generate acceleration measurements along a plurality of axes; and
a processor coupled to the motion sensor, the processor configured to:
detect a head movement event based on the acceleration measurements, the head movement event indicating cough head movement by a user;
detect a vocal activity event based on the acceleration measurements, the vocal activity event indicating cough vocal activity by the user;
detect a cough event in response to detection of the head movement event and the vocal activity event; and
output the cough event.

2. The device of claim 1 wherein the device is a multi-sensor device that includes the motion sensor and the processor.

3. The device of claim 1 wherein the motion sensor includes a bone conduction accelerometer configured to generate the acceleration measurements.

4. The device of claim 1 wherein the processor is configured to:
determine acceleration values based on the acceleration measurements, the acceleration values being indicative of the acceleration measurements along the plurality of axes, the head movement event being detected based on the acceleration values, the vocal activity event being detected based on the acceleration values.

5. The device of claim 4 wherein the processor is configured to:
filter the acceleration values with a band pass filter; and
down sample the filtered acceleration values.

6. The device of claim 5 wherein the processor is configured to:
determine time domain features of the down sampled filtered acceleration values; and
detect the head movement event in case the time domain features satisfy one or more determined conditions.

7. The device of claim 6 wherein the time domain features include at least one of a root mean square calculation, an auto correlation calculation, an envelope calculation, and a zero crossing rate calculation.

8. The device of claim 4 wherein the processor is configured to:
convert the acceleration values to a frequency domain; and
determine magnitude values of the acceleration values in the frequency domain.

9. The device of claim 8 wherein the processor is configured to:
determine frequency domain features of the magnitude values of the acceleration values in the frequency domain; and
detect the vocal activity event in case the frequency domain features satisfy one or more determined conditions.

10. The device of claim 9 wherein the frequency domain features include at least one of an energy calculation, a spectral centroid calculation, a spectral spread calculation, and a maximum energy bin calculation.

11. The device of claim 9 wherein the frequency domain features include a maximum energy bin calculation.

12. The device of claim 1 wherein the processor is configured to:
validate the cough event in case the cough event is detected for a determined time duration and detected a determined amount of time after a previous cough event.

13. The device of claim 1 wherein the processor is configured to:
determine acceleration values based on the acceleration measurements;
filter the acceleration values with a band pass filter;
down sample the filtered acceleration values;
determine time domain features of the down sampled filtered acceleration values; and
detect the head movement event based on the time domain features.

14. The device of claim 13 wherein the time domain features include an envelope calculation, and the head movement event is detected with a neural network.

15. The device of claim 1 wherein the processor is configured to:
determine acceleration values based on the acceleration measurements;
convert the acceleration values along the axis to a frequency domain;
determine magnitude values of the acceleration values in the frequency domain; and
detect the vocal activity event based on the magnitude values.

16. The device of claim 15 wherein the vocal activity is detected with a neural network.

17. A method, comprising:
generating, by a motion sensor, acceleration measurements along a plurality of axes;
detecting, by a processor coupled to the motion sensor, a head movement event based on the acceleration measurements, the head movement event indicating cough head movement by a user;
detecting, by the processor, a vocal activity event based on the acceleration measurements, the vocal activity event indicating cough vocal activity by the user;
detecting, by the processor, a cough event in response to detecting the head movement event and the vocal activity event; and
outputting, by the processor, the cough event.

18. The method of claim 17, further comprising:
validating, by the processor, the cough event in case the cough event is detected for a determined time duration and detected a determined amount of time after a previous cough event.

19. A multi-sensor device, comprising:
an accelerometer configured to generate acceleration measurements; and
a processor coupled to the accelerometer, the processor configured to:
determine time domain features of the acceleration measurements;
detect a head movement event based on the time domain features, the head movement event indicating cough head movement by a user;
convert the acceleration measurements to a frequency domain;
detect a vocal activity event based on the acceleration measurements in the frequency domain, the vocal activity event indicating cough vocal activity by the user;
detect a cough event in response to detection of the head movement event and the vocal activity event; and
output the cough event.

20. The multi-sensor device of claim 19 wherein the processor is configured to:
validate the cough event in case the cough event is detected for a determined time duration and detected a determined amount of time after a previous cough event.
